(19) United States
(12) Patent Application Publication
(10) Pub. No.: US 2023/0325487 A1
(43) Pub. Date: Oct. 12, 2023

(54) PRODUCTION OF THERAPEUTICS POTENTIAL MESENCHYMAL STEM CELLS

(71) Applicant: CRYOCORD SDN BHD, Cyberjaya (MY)

(72) Inventors: Ming Foong CHAI, Cyberjaya (MY); Zhi Xin LEE, Cyberjaya (MY); Vijayendran GOVINDASAMY, Cyberjaya (MY); Soon Keng CHEONG, Cyberjaya (MY); Khong Lek THEN, Cyberjaya (MY)

(73) Assignee: CRYOCORD SDN BHD, Cyberjaya (MY)

(21) Appl. No.: 18/208,247

(22) Filed: Jun. 9, 2023

Related U.S. Application Data
(62) Division of application No. 16/653,350, filed on Oct. 15, 2019, now Pat. No. 11,144,629.

(30) Foreign Application Priority Data
Jun. 12, 2019 (MY) PI 2019003325

(51) Int. Cl. G06F 21/36; G06F 21/45

(52) U.S. Cl. CPC G06F 21/36; G06F 2221/2133; G06F 21/45

(57) ABSTRACT

The present invention relates to production of therapeutic potential WJ-MSCs for clinical purposes. The WJ-MSCs cultured in optimal culture conditions for production of clinical grade WJ-MSCs wherein 4 different culture media which are Media A, Media B, Media C and Media D used to produce 4 types of WJ-MSCs with different therapeutic potential. The WJ-MSCs harvested from Media A has therapeutic potential related to immune, wound healing and cell migration and the WJ-MSCs harvested from Media B has therapeutic potential related to localization, cell proliferation and cell migration. Meanwhile, the WJ-MSCs harvested from Media C has therapeutic potential related to organ development and osteogenesis and the WJ-MSCs harvested from Media D has therapeutic potential related to tissue development, cell signaling and localization.

### FIELD OF THE INVENTION

The present invention relates to production of therapeutic potential of mesenchymal stem cells (MSCs) specifically MSCs obtained from Wharton's jelly (WJ-MSCs) using culture composition and method of diagnosis of the therapeutic potential thereof which is done by analysing the expression of set of genes using next generation sequencing. The invention is brought about from the belief of ‘We are what we eat’ and hence ‘Cells are what cells eat’.

### BACKGROUND OF THE INVENTION

Mesenchymal stem cells (MSCs) are cells with special self-renewal capacity and capable of differentiation into mesenchymal tissues such as bone, cartilage and fat. There are also studies suggesting that MSCs have tendency to differentiate into other lineages but this is largely depends on the origin where they are isolated. They have immune-modulatory and anti-inflammatory effects and also known as a regenerative medicine. MSCs are generally defined as clonogenic cells capable of both self-renewal and multi-lineage differentiation. Post-natal MSCs have been isolated from various tissues, including bone marrow, adipose, skin, retina, dental tissues and umbilical cord. However, MSCs's immune properties appear to be more robustly expressed and functional with umbilical cord specifically Wharton's Jelly (WJ) tissues isolated from umbilical cord in comparison with other tissue derived MSCs (i.e. bone marrow or adipose).

This is because due to the primitive age of the WJ which suggest that MSCs harvested from this tissue will exhibit a much more proliferative, immunosuppressive, and even therapeutically active stem cells than those isolated from other tissue sources such as the bone marrow or adipose. In addition, MSCs from the umbilical cord specifically WJ tissues isolated from umbilical cord are easily accessed and obtained compared to bone marrow and embryonic stem cells. WJ-MSCs have a high proliferation valency and they do not turn into teratogenic or carcinogenic cells in case of transplantation.

Typically, the selection of culture media and its supplements is a critical prerequisite for the maintenance and expansion of MSCs in vitro. The right culture condition is important as it influence the behavior and function of MSCs in a clinical setup. Further, in view of potential application of WJ-MSCs for clinical medicine, many researches have been conducted to optimize the WJ-MSCs expansion protocols to meet the demand of the cells for therapeutic applications. This also focuses on maintaining the functional capabilities of the cells along with the phenotypic characteristics in a cost effective manner.

WO2011101834 A1 discloses a media comprising about 50% KO-DMEM and about 50% alpha MEM media, optionally supplemented with at least one of the supplements selected from a group comprising human serum albumin, growth factors, platelet lysate, amino acids and bioactive agents.

US2011312091 A1 discloses a method of isolating, purifying and culturally expanding of a population of human pluripotent stem cells. It also discloses grow media I consist of 50-70% DMED/F12 and 30-50% MCDB-201, supplemented with 2-10% serum, 8-10 mol/L dexamethasone, 10-50 mg/mL insulin-transferrin-selenium (ITS), 0.1-10 mmol/L glutamine, 1-100 ng/mL human epidermal growth factor (hEGF) and 1-100 ng/mL basic fibroblast growth factor (bFGF). Meanwhile, grow media II consist of 50-70% DMED/F12 and 30-50% MCDB-201, supplemented with 0.1-5% (W/V) human serum albumin (HSA), 1-100 μg/mL linolenic acid, 1-100 μg/mL linoleic acid, 0.1-5% non-essential amino acid, 10<−8>mol/L dexamethasone, 10-50 mg/mL insulin-transferrin-selenium (ITS), 0.1-10 mmol/L glutamine, 1-100 ng/mL human epidermal growth factor (hEGF) and 1-100 ng/mL basic fibroblast growth factor (bFGF).

WO2017132358 A1 discloses a culture media comprising glucose and glutamine, for example Dulbecco's Modified Eagle Media (DMEM), which generally comprises glucose (low or high) and glutamine (e.g., Gibco™ GlutaMAX™), as well as human platelet lysate (e.g., pooled human platelet lysate), and heparin.

Till to date, there is inconsistency among laboratories with regards to the types of culture media and the additional factors for the successful isolation and expansion of WJ-MSCs. This results in inconsistency in the cell production affects tremendously on the therapeutic potential especially during ex-vivo experiments and clinical trials. Further, despite many isolation methods as well as culturing composition for culturing and expanding WJ-MSCs disclosed in the prior art documents, none of it actually demonstrates genetic implications as a result of media compositions and therapeutic specificity such as the ability of the cells to be used for a specific disease or disorder.

Thus, the present invention, overcomes the inconsistency by setting optimal culture conditions for effective clinical-grade production of large number of WJ-MSCs and diagnosis of therapeutic targets of WJ-MSCs by analysing the expression of genes from results of next generation sequencing. Results of this study are highly reproducible and consistent, making them useful for quality control in cell production under Current Good Manufacturing Practice (cGMP) for clinical purposes.

### SUMMARY OF THE INVENTION

The present invention discloses a process of producing therapeutic potential WJMSCs. The process for producing therapeutic potential Wharton's Jelly mesenchymal stem cells (WJ-MSCs) comprising the steps of: (i) isolating and culturing WJ-MSCs to produce primary cell lines; (ii) expanding primary cell lines obtained in step (i) from passage 0 to passage 2; (iii) cryopreserving cells from passage 2 in a cryopreservation tank to produce cryopreserved cells; (iv) thawing the cryopreserved cells obtained from step (iii); (v) expanding the cells obtained from step (iv) to passage 6 in four different complete culture media which are (i) Media A (ii) Media B; (iii) Media C and (iv) Media D; and (vi) harvesting the cells obtained from step (v) to obtain therapeutic potential WJ-MSCs wherein the WJMSCs harvested from each culture media have different therapeutic potential. The culture media composition of Media A comprising of DMEM basal media in a range of 84 to 96%, platelet lysate in a range of 3 to 10%, antibiotic and antimycotic Gibco™ in a range of 0.5 to 3% and glutamax Gibco™ in a range of 0.5 to 3% wherein the culture media induce the cells to express genes mainly related to immunology and wound healing and cell migration towards neural The culture media composition of Media B comprising of DMEM-KO basal media Gibco™ in a range of 84 to 96%, platelet lysate in a range of 3 to 10%, antibiotic and antimycotic Gibco™ in a range of 0.5 to 3% and glutamax Gibco™ in a range of 0.5 to 3% wherein the culture media induce the cells to express genes related to localization, cell proliferation and cell migration. The culture media composition of Media C comprising of SFM Xeno Free basal media Gibco™ in a range of 93 to 98%, SFM Xeno Free supplement Gibco™ in a range of 1%, antibiotic and antimycotic Gibco™ in a range of 0.5 to 3% and glutamax Gibco™ in a range of 0.5 to 3% wherein the culture media induce the cells to express genes related to organ development and osteogenesis. The culture media composition of Media D comprising of SFM Xeno Free basal media Gibco™ in a range of 88 to 97%, SFM Xeno Free supplement Gibco™ in a range of 1%, platelet lysate in a range of 1 to 5%, antibiotic and antimycotic Gibco™ in a range of 0.5 to 3% and glutamax Gibco™ in a range of 0.5 to 3% wherein the culture media induce the cells to express genes related to tissue development, cell signaling and localization.

Additional aspects, features and advantages of the invention will become apparent to those skilled in the art upon consideration of the following detailed description of preferred embodiments of the invention in conjunction with the drawings listed below.

### BRIEF DESCRIPTION OF THE ACCOMPANYING FIGURES

The accompanying figures are included to provide a further understanding of the present disclosure and are incorporated in and constitute a part of this specification. The figures illustrate exemplary embodiments of the present disclosure and, together with the description, serve to explain the principles of the present disclosure.

FIG. 1 displays a table for the average of cell population doubling time obtained by using culture media A, B, C and D in accordance with the present invention.

FIG. 2 displays cell differentiation assay for adipocytes, osteocytes and chondrocytes obtained using culture media A, B, C and D in accordance with the present invention.

FIG. 3 displays flow cytometry results for the immunophenotyping of cell surface markers obtained using culture media A, B, C and D in accordance with the present invention.

FIG. 4 displays work flow of next generation sequencing and pre analysis of raw data file in accordance with the present invention.

FIG. 5 displays a uniquely expressed number of genes compared among culture media A, B, C and D in Venn Diagram in accordance with the present invention.

### DETAILED DESCRIPTION OF THE INVENTION

Detailed description of preferred embodiments of the present invention is disclosed herein. It should be understood, however, that the embodiments are merely exemplary of the present invention, which may be embodied in various forms. Therefore, the details disclosed herein are not to be interpreted as limiting, but merely as the basis for the claim and for teaching one skilled in the art of the invention.

An object of the invention is to produce specific therapeutic potential of MSCs.

Another object of the invention is to set optimal culture conditions for clinical grade production of MSCs under cGMP.

Another object of the present invention is to diagnose therapeutic targets of WJ-MSCs by analysing the expression of genes using next generation sequencing. As indicated, the present invention provides an extract of WJ-MSCs as a source of rapidly proliferating cell population which is cultured in optimal culture conditions for production of clinical grade WJ-MSCs wherein 4 different culture media are used to produce 4 types of WJ-MSCs with different therapeutic potential.

The present invention also solve the inconsistency problem by setting an optimal culture conditions for the production of clinical grade WJ-MSCs in a cost and time effective manner. The results of the present invention are highly reproducible and consistent making the WJ-MSCs useful for in vivo as well as in vitro manipulation without losing their vigour and chromosomal stability.

Further objects and advantages of the invention will be brought out in the following portions of the specification, wherein the detailed description is for the purpose of fully disclosing preferred embodiments of the invention without placing limitations thereon. The preferred embodiments will now be described in detail in accordance with the attached figures.

### Example 1: Isolation and Culturing of WJ-MSCs

All umbilical cord specimens used in these inventions were donor consented for research purposes Human mesenchymal stem cells were isolated from human umbilical cord specifically from Wharton's jelly by using enzymatic digestion method. Briefly, umbilical cords were sliced into small pieces and incubated with 40 ml of 1% collagenase I digestive solution in a shaking incubator at a temperature of 37° C. for a period of 2 hours. After 2 hours, the mixture of digestive solution was centrifuged at 600 Relative Centrifugal Force (RCF) for a period of 10 minutes. Supernatant were discarded and pellet were washed with Phosphate-buffered saline (PBS). The pellet was centrifuged again at 600 RCF for a period of 10 minutes. The final supernatant was discarded and pellet was re-suspended with a standard culture media. The culture media comprises of Dulbecco's Modified Eagle's media (DMEM), human platlet Lysate in the range of 3-10% of the final volume, antibiotic-antimycotic in the range of 0.5-3% of the final volume and glutamax in the range of 0.5-3% of the final volume. The culture media with cells were seeded into 175 cm² flask and incubated at a temperature of 37° C., in 5% CO² incubator for a period of 48 hours. After 48 hours, non-adherent cells were removed from the flask by replenishing the media with a new one. Subsequently, media changes were performed for every 3-4 days, until the cells reaches confluency. At 80-90% confluence, WJ-MSCs were trypsinized and reseeded in a cell destiny of 2500/cm² into a 175 cm² tissue culture flask. The primary cell lines (PO) derived from the umbilical cord (Wharton's jelly) were uniquely identified as RUCM 0619, RUCM 3008 and RUCM 3678. These cells were used for the subsequent experiments.

### Example 2: Expansion of WJ-MSCs from Passage 0-Passage 2

The T-175 cm² culture flasks containing PO cells were transferred into cleaned and sterilized Biological Safety Cabinet (BSC). PBS was used for rinsing upon removing all conditioned media from culture flasks via serological pipettes. The flasks were left for a period of 1 minute and the PBS was discarded into a waste beaker and later added with 10 mL of disassociate enzyme into flask and incubated at a temperature of 37° C. in 5% humidified CO₂ incubator for less than a period of 10 minutes. Cells were observed under inverted microscope for round and floating cells to confirm complete cells detachment. Cell suspension was transferred into a new 50 mL centrifuge tube and centrifuged at 600 RCF for 10 minutes at a temperature of 20° C.±2° C. The supernatant was discarded into waste beaker and 20 mL of Conditioned Culture Media (CCM) was added into the tube to re-suspend the pellet. After performing cell count, the cells were cultured into T175 cm² culture flasks for passage 1. The cells were observed under inverted microscope for every 2 days until the cells reach 80±5% confluency. Upon reaching 80±5% confluency, cells were then sub-cultured to passage 2 with the same process as mentioned above. An appropriate volume of Cell Freezing Media (CFM), with a mixture of Human Serum Albumin and Dimethyl Sulfoxide (DMSO) was prepared to cryo-preserve the cells.

### Example 3: Complete Culture Media Composition Used in this Study

Human mesenchymal stem cells from P2 from the cryopreservation tank were thawed and expanded to P6 in four different culture media, namely Media A, Media B, Media C and Media D containing combinations of platelet lysate, supplements and serum free components. The media composition of the four different media is listed in Table 1.

TABLE 1
Composition of complete culture media

| | Range of Media Composition |
|---|---|
| | in Final Volume |
| Media A | |
| DMEM basal media | 84.0-96.0% |
| Platelet Lysate | 3.0-10.0% |
| Antibiotic and Antimycotic | 0.5-3.0% |
| Glutamax | 0.5-3.0% |
| Media B | |
| DMEM-KO basal media | 84.0-96.0% |
| Platelet Lysate | 3.0-10.0% |
| Antibiotic and Antimycotic | 0.5-3.0% |
| Glutamax | 0.5-3.0% |
| Media C | |
| SFM Xeno Free basal media | 93.0-98.0% |
| SFM Xeno Free supplement | 1.0% |
| Antibiotic and Antimycotic | 0.5-3.0% |
| Glutamax | 0.5-3.0% |
| Media D | |
| SFM Xeno Free basal media | 88.0-97.0% |
| SFM Xeno Free supplement | 1.0% |
| Platelet Lysate | 1.0-5.0% |
| Antibiotic and Antimycotic | 0.5-3.0% |
| Glutamax | 0.5-3.0% |

### Example 4: Growth Kinetics Assay

The growth kinetics was determined by plating 5000 cells/cm² from WJ-MSCs per T75 cm² culture flask. Three replicates were performed for each passage. Cells were detached by trypsinization after reaching confluency of 90%. Growth kinetics was analyzed by calculating population doubling (PD) time.

The PD time was obtained using the formula:

Population Doubling Time=Time×Log 2/[Log(Final Concentration)−Log(Initial Concentration)]

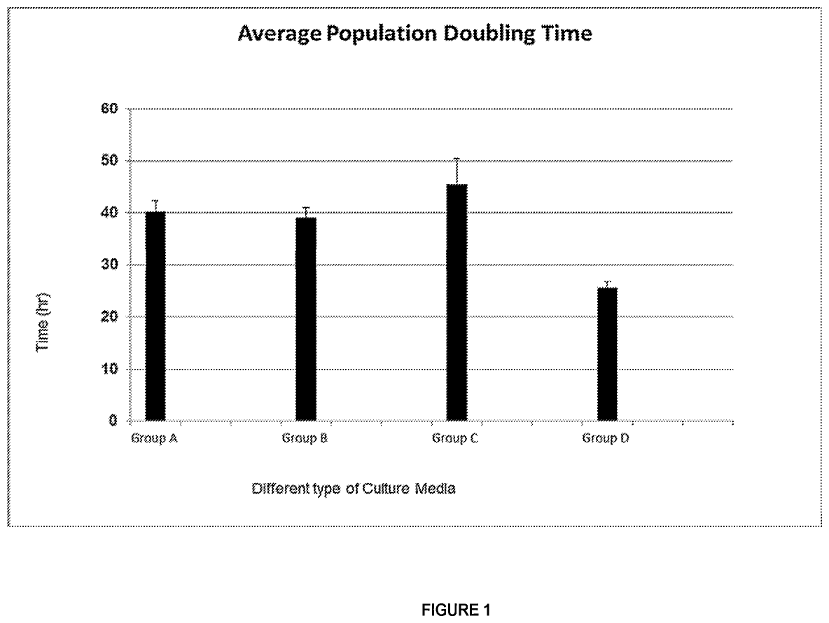

The average population doubling time is tabulated in FIG. 1. The WJ-MSCs cultured in Media A, B and C showed a stable PD time whereas media D showed the lowest PD.

### Example 5: Differentiation into Mesoderm Lineage

All samples from different condition of culture media (Media A, Media B, Media C and Media D) with 500,000 cells were seeded into a well of 6 wells. The 6 wells plates were incubated in a CO₂ incubator for a period of 2-3 days. After the cells were reached 80% confluency, culture media were removed and substituted with three types of differentiation media, adipogenesis, osteogenesis and chondrogenesis (StemPro, Invitrogen). The differentiation media were changed every 3-4 days for a period of 21 days. At 21 days, differentiation media were removed and rinsed with PBS. The cells were fixed using 3.7% paraformaldehyde for further staining procedure. The details are summarized below:

a) Alcian Blue Stain for In Vitro Chondrogenesis

The fixed cells were stained using paraffin and then the cells were embedded for tissue sectioning. A thin slice of sectioning tissue was mounted on microscope. Alcian Blue stain was used to confirm the formation of proteoglycan component of cellular matrix.

b) Alizarin Red S Stain for In Vitro Osteogenesis

The fixed cells were stained using Alizarin Red S solution (Merck). Alizarin Red S Stain was used to confirm the formation of calcium deposition.

c) Oil Red O Stain for In Vitro Adipogenesis

The fixed cells were stained using Oil Red O stain Oil red O stain to confirm the lipid droplet formation.

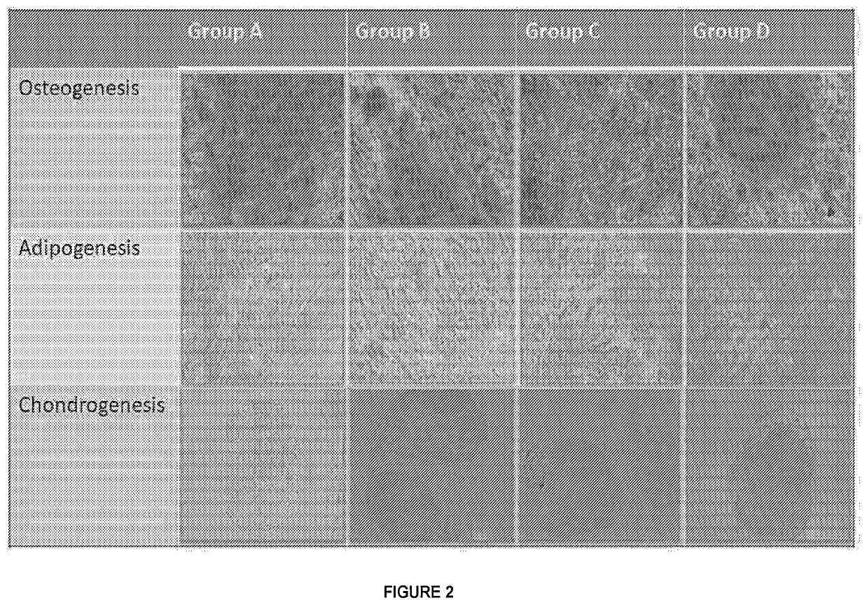

The cell differentiation adipogenesis, osteogenesis and chondrogenesis in different culture media (Media A, Media B, Media C and Media D) are shown in FIG. 2.

### Example 6: Analysis of Surface Antigen Markers by Flow Cytometry

BD stem flow human MSCs analysis kit was used for the identification of MSCs surface markers. All samples from different media (Media A, Media B, Media C and Media D) were stained as per the manufacturer protocol of the BD stem flow human MSCs analysis kit. All stained samples were acquired using a BD FACS via flow cytometry and at least 10,000 events were collected. The data were analyzed using Cell Quest software BD Bioscience.

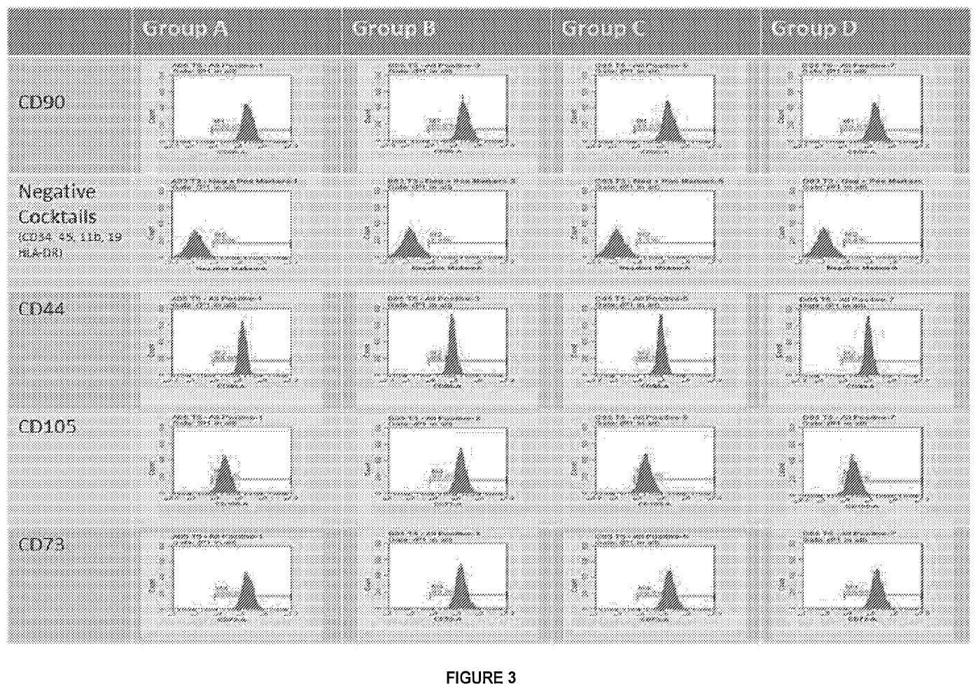

FIG. 3 shows flow cytometry results for the immune phenotyping of cell surface markers obtained using culture media A, B, C and D. Immuno-phenotyping of stem cells derived from WJ-MSCs showed that the cells were negative for hematopoietic markers CD11b, CD19, CD34, CD45 and HLA-DR, whereas more than 90% of cells were positive for mesenchymal stem cell markers CD44, CD73, CD90 and CD105.

### Example 7: Gene Expression Study by Using Next Generation Sequencing (NGS)

Total RNA was extracted from cells obtained from 4 different medias (Media A, Media B, Media C and Media D) and 2 replicates using RNeasy Mini Kit (Qiagen) according to manufacturer instruction. Extracted total RNA was quantified by using Bioanalyser 2100 Eukaryote 6000 Nano Chip. RNA with integrity RIM>8 were stored at a temperature of −80° C. for library preparation. cDNA was prepared by using TruSeq Straded mRNA library kit (illumina). The cDNA was sequenced using Hiseq 4000 with 150 bp paired end reads up 100 million reads on e-processing of raw data, sequence annotation and identification of differentially expressed (DE) genes.

FastQ formatted sequencing data were generated from the next generation sequencing. Fast Q sequencing data were undergo a series of quality control include quality score of each bases and each sequence, reads filtering and adapter removal. Fastx-toolkit (version 0.0.14) was used to check quality and reads filtering. Trimmomatic was used for the removal of adapter from each sequence. The clean reads were mapped to human reference genome GRCH 38 using STAR. EdgeR was used to normalization and differentially expression of total mapped reads.

Gene Ontology and Enrichment Analysis

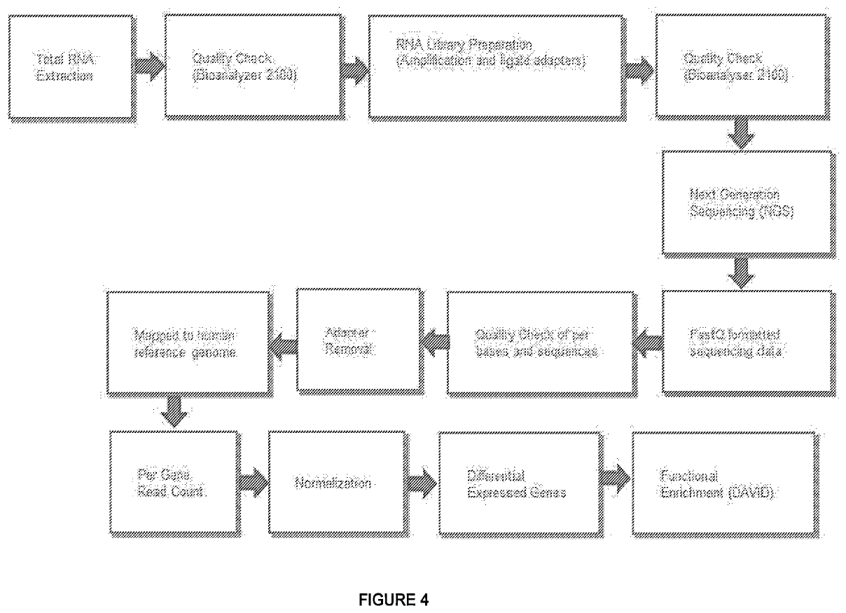

In order to identify statistically significant genes between 4 different medias (Media A, Media B, Media C and Media D), the genes must have log 2 (fold change>2 and p-value<0.05. The uniquely expressed genes of one media to another media (eg. Media A vs Media B, Media C and Media D) were submitted to Database for Annotation, Visualization and Integrated Discovery (DAVID) web server for analysis. FIG. 4 displays work flow of next generation sequencing and pre analysis of raw data file in accordance with the present invention.

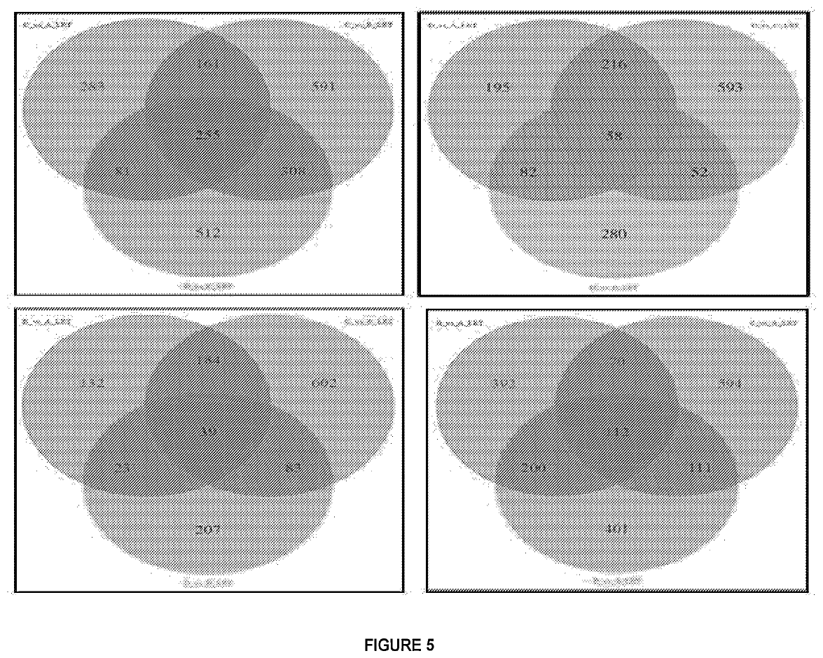

Overall, based on the next generation sequencing data, a total of 255, 58, 39 and 112 uniquely (novel) expressed genes (p<0.05) were found in media A, B, C, D respectively. FIG. 5 displays a uniquely expressed number of genes compared among culture media A, B, C and D in Venn Diagram. These novel genes were further analyzed for their biological functionality. Database for Annotation, Visualization and Integrated Discovery (David) analysis revealed novel biological of stem cells cultured in various media.

Table 2 displays the list of genes identified toward specific biological function in culture media A, B, C and D using DAVID analysis.

Culture Media A:

| | Biological | | | |
|---|---|---|---|---|
| | Process Main | Sub- | | |
| No | Category | category | P Value | List of Genes |
| 1 | Cell Migration | Ameboidal | 0.0063 | HTR2B, CEACAM1, |
| | | (able to alter | | CTSH ERBB4, |
| | | its shape) | | HDAC9, NOV, |
| | | | | SEMA6A |
| | | Neural Crest | 0.03 | HTR2B, ERBB4, |
| | | | | SEMA6A |
| 2 | Immune | T cell | 0.011 | CEACAM1, CTSH, |
| | | mediated | | CRTAM |
| | | cytotoxicity | | |
| 3 | Wound | Response to | 0.019 | CD177, CD36, |
| | (Towards wound | wounding | | CEACAM1, ERBB4, |
| | healing) | | | HBD, NOV, ODAM, |
| | | | | SPP1, TMEFF2 |
| | | Wound | 0.022 | CD177, CD36, |
| | | Healing | | CEACAM1 ERBB4, |
| | | | | HBD, NOV, ODAM, |
| | | | | TMEFF2 |

Culture Media B:

| | Biological | | | |
|---|---|---|---|---|
| | Process Main | | | |
| No | Category | Sub-category | P Value | List of Genes |
| 1 | Localization | Localization | 0.047 | CD163L1, CD274, |
| | (Cells able to | | | EPPK1, HBEGF, |
| | perform | | | MMP9, NPPB, |
| | localize and | | | PLN, KCNMB1, |
| | integrate with | | | KCNN3, KCNU1, |
| | host) - | | | SLC12A5, |
| | indicating | | | SLC7A2, TIE1 |
| | more of Graft | Regulation of | 0.0026 | CD274, EPPK1, |
| | versus Host | localization | | HBEGF, MMP9, |
| | (GvHD) | | | NPPB, PLN, |
| | | | | KCNMB1, KCNN3, |
| | | | | KCNU1, TIE1 |
| | | Ion | 0.0012 | MMP9, PLN, |
| | | transmembrane | | KCNMB1, KCNN3, |
| | | transport | | KCNU1, SLC12A5, |
| | | | | SLC7A2, |
| 2 | Cell | Smooth muscle | 0.0089 | CNN1, HBEGF, |
| | Proliferation | | | MMP9 |
| | (Muscle | | | |
| | development) | | | |
| 3 | Cell Migration | Regulation of | 0.000046 | EPPK1, HBEGF, |
| | (Towards wound | keratinocyte | | MMP9 |
| | healing) | migration | | |
| | | Keratinocyte/ | 0.00013 | EPPK1, HBEGF, |
| | | fibro blast | | MMP9 |
| | | migration | | |

Culture Media C:

| | Biological | | | |
|---|---|---|---|---|
| | Process Main | | | |
| No | Category | Sub-category | P Value | List of Genes |
| 1 | General Organ | Skeletal system | 0.0000075 | MAF, RSPO2, |
| | development | development | | ACVR2A, BMP6, |
| | | | | COMP, CMKLR1, |
| | | | | CHI3LI, CHRDL2, |
| | | | | COL12A1, FGFR2, |
| | | | | GSC, GDF10, |
| | | | | IGF1, MGP, |
| | | | | PAPPA2, PTHLH, |
| | | | | RBP4 |
| | | Connective | 0.0012 | MAF, PPARGC1A, |
| | | tissue | | RSPO2, BMP6, |
| | | development | | COMP, CHI3LI, |
| | | | | CHRDL2, MGP, |
| | | | | PTHLH |
| | | Cartilage | 0.0012 | MAF, RSPO2, |
| | | development | | BMP6, COMP, |
| | | | | CHI3LI, CHRDL2, |
| | | | | MGP, PTHLH |
| 2 | Bone | Bone | 0.0084 | RSPO2, ACVR2A, |
| | Development | mineralization | | BMP2, FGFR2, |
| | (Osteogenesis) | | | IGF1 |
| | | Positive | 0.00086 | ACVR2A, BMP6, |
| | | regulation of | | GDF10, INHBE, |
| | | pathway- | | MSTN |
| | | restricted | | |
| | | SMAD protein | | |
| | | phosphorylation | | |

Culture Media D:

| | Biological | | | |
|---|---|---|---|---|
| | Process Main | | | |
| No | Category | Sub-category | P Value | List of Genes |
| 1 | Tissue | Tissue | 0.018 | APCDD1, EPHA7, |
| | development | development | | HOPX, AQP3, |
| | | | | CRABP2, FGF20, |
| | | | | GAP43, SOSTDC1, |
| | | | | TCF7 |
| | | Epidermis | 0.021 | APCDD1, AQP3, |
| | | development | | CRABP2, SOSTDC1 |
| 2 | Cell signaling | Cell-cell | 0.0078 | APCDD1, CRHBP, |
| | | signaling | | FGF20, NKD2, |
| | | | | NPTX1, SOSTDC1, |
| | | | | SNAP25, SYTL2, |
| | | | | TCF7 |
| | | Exocytosis | 0.044 | CRABP2, NKD2, |
| | | | | SNAP25, SYTL2 |
| 3 | Localization | Regulation of | 0.017 | CLIC6, CCDN7, |
| | (Cells able | localization | | CRHBP, DOCK2, |
| | to perform | | | FGF20, NKD2, |
| | localize and | | | NOS1, KCNH5, |
| | integrate with | | | RGN, SNAP25, |
| | host) - | | | SYTL2 |
| | indicating | Vesicle fusion | 0.02 | NKD2, SNAP25, |
| | more of Graft | | | SYTL2 |
| | versus Host | | | |
| | (GvHD) | | | |

WJ-MSCs cultured in media A highly expressed genes such as HTR2B, CEACAM1, CTSH, ERRB4, HDAC9, NOV, SEMA6A, CRTAM, CD177, CD36, HBD, ODAM, SPP1 and TMEFF2 which collectively indicating biological process related to immune, wound and cell migration (towards neural crest). The same populations of cells expressed differently with the following genes were highly presence in Media B: CD163L1, CD274, EPPK1, HBEGF, MMP9, NPPB, PLN, KCNMB1, KCNN3, KCNU1, SLC12A5, SLC7A2 TIE1 and CNN1. These genes are responsible for localization, cell proliferation and cell migration. Interestingly, WJ-MSCs cultured in Media C shows propensity towards organ development and osteogenesis. This biological process characterization was based on the following genes MAF, RSPO2, ACVR2A, BMP6, COMP, CMKLR1, CHI3L1, CHRDL2, COL12A1, FGFR2, GSC, GDF10, IGF1, MGP, PAPPA2, PTHLH, RBP4, BMP2, INHBE, MSTN and PPARGC1A. WJ-MSCs cultured in Media D shows a higher propensity toward tissue development, cell signaling and localization. EPHA7, HOPX, AQP3, CRABP2, FGF20, GAP43, TCF7 APCDD1, SOSTDC1 CRHBP, NKD2, NPTX1, SNAP25, SYTL2, CLIC6, CCDN7, DOCK2, NOS1, KCNH5 and RGN.

The culture media of the present invention provides a solution to produce clinical grade MSCs with therapeutic potential. It also discloses a method to diagnose therapeutic targets of WJ-MSCs by analysing the expression of genes using next generation sequencing platform. This also focuses on maintaining the functional capabilities of the cells along with the phenotypic characteristics in a cost effective manner.

The present invention also solve the inconsistency problem in cell production specifically during clinical trials and ex-vivo experiments by setting an optimal culture conditions for the production of clinical grade WJ-MSCs in a cost and time effective manner. The results of the present invention are highly reproducible and consistent making the WJ-MSCs useful for in vivo as well as in vitro manipulation without losing their vigour and chromosomal stability.

It is to be understood that the terminology used herein is for the purpose of describing particular example embodiments only and is not intended to be limiting. As used herein, the singular forms “a”, “an” and “the” may be intended to include the plural forms as well, unless the context clearly indicates otherwise.

The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or media thereof.

The method steps, processes, and operations described herein are not to be construed as necessarily requiring their performance in the particular order discussed or illustrated, unless specifically identified as an order of performance. It is also to be understood that additional or alternative steps may be employed. The use of the expression “at least” or “at least one” suggests the use of one or more elements, as the use may be in one of the embodiments to achieve one or more of the desired objects or results.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions or examples, which are included to enable a person having ordinary skill in the art to make and use the disclosure when combined with information and knowledge available to the person having ordinary skill in the art.

1. A process for producing therapeutic potential Wharton's Jelly mesenchymal stem cells (WJ-MSCs) comprising the steps of:
i) isolating and culturing WJ-MSCs to produce primary cell lines;
ii) expanding primary cell lines obtained in step (i) from passage 0 to passage 2;
iii) cryopreserving cells from passage 2 in a cryopreservation tank to produce cryopreserved cells;
iv) thawing the cryopreserved cells obtained from step (iii);
v) expanding the cells obtained from step (iv) to passage 6 in at least one complete culture media; and
vi) harvesting the cells obtained from step (v) to obtain therapeutic potential WJMSCs wherein the complete culture media is selected from a group that consists of:
(A) DMEM basal media in a range of 84 to 96%, platelet lysate in a range of 3 to 10%, antibiotic and antimycotic in a range of 0.5 to 3% and glutamax in a range of 0.5 to 3%,
(B) DMEM-KO basal media in a range of 84 to 96%, platelet lysate in a range of 3 to 10%, antibiotic and antimycotic in a range of 0.5 to 3% and glutamax in a range of 0.5 to 3%,
(C) SFM Xeno Free basal media in a range of 93 to 98%, SFM Xeno Free supplement in a range of 1%, antibiotic and antimycotic in a range of 0.5 to 3% and glutamax in a range of 0.5 to 3%, and
(D) SFM Xeno Free basal media in a range of 88 to 97%, SFM Xeno Free supplement in a range of 1%, platelet lysate in a range of 1 to 5%, antibiotic and antimycotic in a range of 0.5 to 3% and glutamax in a range of 0.5 to 3%,
wherein the WJ-MSCs harvested from each culture media have different therapeutic potential owing to differentially expressed genes.

2. The process for producing therapeutic potential Wharton's Jelly mesenchymal stem cells (WJ-MSCs) of claim 1, wherein the differentially expressed genes are analyzed by Next Generation Sequencing (NGS).

3. The process for producing therapeutic potential Wharton's Jelly mesenchymal stem cells (WJ-MSCs) of claim 1, wherein DMEM basal media deferentially expresses genes selected from HTR2B, CEACAM1, CTSH, ERRB4, HDAC9, NOV, SEMA6A, CRTAM, CD177, CD36, HBD, ODAM, SPP1 and TMEFF2; and wherein the said genes have therapeutic potential related to immune, wound and cell migration.

4. The process for producing therapeutic potential Wharton's Jelly mesenchymal stem cells (WJ-MSCs) of claim 3, wherein the differentially expressed genes are HTR2B, CEACAM1, CTSH, ERBB4, HDAC9, NOV, and SEMA6A; and have therapeutic potential related to cell migration.

5. The process for producing therapeutic potential Wharton's Jelly mesenchymal stem cells (WJ-MSCs) of claim 3, wherein the differentially expressed genes are HTR2B, ERBB4, SEMA6A, CEACAM1, CTSH, and CRTAM; and have therapeutic potential related to immune.

6. The process for producing therapeutic potential Wharton's Jelly mesenchymal stem cells (WJ-MSCs) of claim 3, wherein the differentially expressed genes are CD177, CD36, CEACAM1, ERBB4, HBD, NOV, ODAM, SPP1, TMEFF2 and have therapeutic potential related to wound healing and wounding response.

7. The process for producing therapeutic potential Wharton's Jelly mesenchymal stem cells (WJ-MSCs) of claim 1, wherein DMEM-KO basal media differentially expresses genes selected from CD163L1, CD274, EPPK1, HBEGF, MMP9, NPPB, PLN, KCNMB1, KCNN3, KCNU1, SLC12A5, SLC7A2, TIE1 and CNN1; and wherein the said genes have therapeutic potential related to cell localization, cell proliferation and cell migration.

8. The process for producing therapeutic potential Wharton's Jelly mesenchymal stem cells (WJ-MSCs) of claim 7, wherein the differentially expressed genes are CD163L1, CD274, EPPK1, HBEGF, MMP9, NPPB, PLN, KCNMB1, KCNN3, KCNU1, SLC12A5, SLC7A2, and TIE1; and have therapeutic potential related to cell localization.

9. The process for producing therapeutic potential Wharton's Jelly mesenchymal stem cells (WJ-MSCs) of claim 7, wherein the differentially expressed genes are CNN1, HBEGF, and MMP9 and have therapeutic potential related to cell proliferation of smooth muscles.

10. The process for producing therapeutic potential Wharton's Jelly mesenchymal stem cells (WJ-MSCs) of claim 7, wherein the differentially expressed genes are EPPK1, HBEGF, and MMP9; and have therapeutic potential related to cell migration.

11. The process for producing therapeutic potential Wharton's Jelly mesenchymal stem cells (WJ-MSCs) of claim 1, wherein SFM Xeno Free basal media of step C) expresses genes selected from MAF, RSPO2, ACVR2A, BMP6, COMP, CMKLR1, CHI3L1, CHRDL2, COL12A1, FGFR2, GSC, GDF10, IGF1, MGP, PAPPA2, PTHLH, RBP4, BMP2, INHBE, MSTN, MGP, and PPARGC1A; and have therapeutic potential related to organ development and osteogenesis.

12. The process for producing therapeutic potential Wharton's Jelly mesenchymal stem cells (WJ-MSCs) of claim 11, wherein the differentially expressed genes are MAF, RSPO2, ACVR2A, BPM6, COMP, CMKLR1, CHI3L1, CHRDL2, COL12A1, FGFR2, GSC, GDF10, IGF1, MGP, PAPPA2, PTHLH, and RBP4; and have therapeutic potential related to organ development of skeletal system.

13. The process for producing therapeutic potential Wharton's Jelly mesenchymal stem cells (WJ-MSCs) of claim 11, wherein the differentially expressed genes are MAF, PPARGC1A, RSPO2, BMP6, COMP, CHI3L1, CHRDL2, MGP, and PTHLH; and have therapeutic potential related to organ development of connective tissue.

14. The process for producing therapeutic potential Wharton's Jelly mesenchymal stem cells (WJ-MSCs) of claim 13, wherein the differentially expressed genes are MAF, RSPO2, BMP6, COMP, CHI3L1, CHRDL2, MGP, and PTHLH; and have therapeutic potential related to organ development of cartilage.

15. The process for producing therapeutic potential Wharton's Jelly mesenchymal stem cells (WJ-MSCs) of claim 11, wherein the differentially expressed genes are RSPO2, ACVR2A, BMP2, BMP6, FGFR2, IGF1, GDF10, INHBE, and MSTN; and have therapeutic potential related to osteogenesis.

16. The process for producing therapeutic potential Wharton's Jelly mesenchymal stem cells (WJ-MSCs) of claim 1, wherein SFM Xeno Free basal media of step D) expresses genes selected from EPHA7, HOPX, AQP3, CRABP2, FGF20, GAP43, TCF7, APCDD1, SOSTDC1, CRHBP, NKD2, NPTX1, SNAP25, SYTL2, CLIC6, CCDN7, DOCK2, NOS1, KCNH5 and RGN; and have therapeutic potential related to related to tissue development, cell signaling and localization.

17. The process for producing therapeutic potential Wharton's Jelly mesenchymal stem cells (WJ-MSCs) of claim 16, wherein the differentially expressed genes are APCDD1, EPHA7, HOPX, AQP3, CRABP2, FGF20, GAP43, SOSTDC1 and TCF7; and have therapeutic potential related to related to tissue development.

18. The process for producing therapeutic potential Wharton's Jelly mesenchymal stem cells (WJ-MSCs) of claim 16, wherein the differentially expressed genes are APCDD1, CRHBP, FGF20, NKD2, NPTX1, SOSTDC1, SNAP25, SYTL2, TCF7, SYTL2 and CRABP2; and have therapeutic potential related to related to cell signaling.

19. The process for producing therapeutic potential Wharton's Jelly mesenchymal stem cells (WJ-MSCs) of claim 16, wherein the differentially expressed genes are CLIC6, CCDN7, CRHBP, DOCK2, FGF20, NKD2, NOS1, KCNH5, RGN, SNAP25, and SYTL2; and have therapeutic potential related to related to cell localization.
